(19) United States
(12) Patent Application Publication
(10) Pub. No.: US 2016/0369217 A1
(43) Pub. Date: Dec. 22, 2016

(54) PLATES FOR CULTURE OF BIOLOGICAL SAMPLES

(71) Applicant: TOYO GOSEI CO., LTD., Ichikawa-shi (JP)

(72) Inventors: Tomoko Jomura, Ichikawa-shi (JP); Takashi Miyazawa, Ichikawa-shi (JP)

(73) Assignee: TOYO GOSEI CO., LTD., Ichikawa-shi (JP)

(21) Appl. No.: 15/116,791

(22) PCT Filed: Feb. 5, 2015
(86) PCT No.: PCT/JP2015/000521

§ 371 Date: Aug. 4, 2016

Related U.S. Application Data
(60) Provisional application No. 61/936,023, filed on Feb. 5, 2014.

(51) Int. Cl. C12M 1/32; C12M 1/12

(52) U.S. Cl. CPC C12M 23/12; C12M 25/06

(57) ABSTRACT

Plates suitable for cultivation of cells to form spheroids are disclosed.

### CROSS-REFERENCE TO RELATED APPLICATIONS

This application is a national phase entry under 35 U.S.C. §371 of International Patent Application PCT/JP2015/000521, filed Feb. 5, 2015, designating the United States of America and published in English as International Patent Publication WO 2015/118873 A1 on Aug. 13, 2015, which claims the benefit under 35 U.S.C. §119(e) of U.S. Provisional Patent Application Ser. No. 61/936,023 filed on Feb. 5, 2014, the disclosure of each of which is hereby incorporated herein in its entirety by this reference.

### TECHNICAL FIELD

An aspect of this application relates to the fields of plates suitable for biological samples such as cell, bacterium, virus and protein. Another aspect of this disclosure relates to the fields of screening methods of which typical applications are drug development and biopsy.

### BACKGROUND

A plate for culturing cells that has a lattice structure is disclosed in U.S. Pat. No. 5,792,653 (issued Aug. 11, 1998).

### BRIEF SUMMARY

A plate relating to an aspect of this disclosure includes a member. The member has a first plane, a second plane, and a plurality of structural objects, each of which has at least one of a bottom in a recess and a top in a protrusion; a first distance from the second plane to a boundary between each of the plurality of objects, the first plane being different from a second distance between the second plane and the at least one of the bottom and the top; and at least five structural objects of the plurality of structural objects are arranged at a third distance from one structural object of the plurality of structural objects.

With regard to the plate, it is preferred that six structural objects of the plurality of structural objects including the at least five structural objects are arranged at the third distance from the one structural object.

The plate can be used for cultivation of cells to form spheroids. It is preferable that the third distance is equal to or greater than 400 micrometers for cultivation of cancer cells, which are to form spheroids having isotropic shapes, or close to them, like human colorectal adenocarcinoma cells (DLD-1). It is more preferable that the third distance is greater than 800 micrometers for cultivation of such cancer cells. On the other hand, it is preferable that the third distance is equal to or smaller than 400 micrometers for cultivation of cancer cells, which are to form spheroids having indefinite shapes, or close to them, like human breast adenocarcinoma cells (SKBR3).

It is preferable that the third distance is equal to or smaller than 400 micrometers for cultivation of normal cells. It is more preferable that the third distance is equal to or smaller than 200 micrometers for cultivation of normal cells.

With regard to the plate, it is preferred that the one structural object and two structural objects of the six structural objects substantially constitute an equilateral triangle.

With regard to the plate, it is preferred that the two structural objects are arranged at positions proximate to each other.

With regard to the plate, it is preferred that the six structural objects substantially constitute a regular hexagon.

A plate relating to an aspect of this disclosure includes a member. The member has a first plane and a second plane. A plurality of first portions and a second portion are formed in the first plane. An affinity of the plurality of first portions to a sample is different from an affinity of the second portion to the sample. At least five first portions of the plurality of first portions are arranged at a third distance from one first portion of the plurality of first portions.

Examples of such sample are chemical compound and biological samples such as cell, bacterium, virus and protein.

With regard to the plate, it is preferred that six first portions of the plurality of first portions including the at least five structural portions are arranged at the third distance from the one first portion.

The plate can be used for cultivation of cells to form spheroids. It is preferable that the third distance is equal to or greater than 400 micrometers for cultivation of cancer cells, which are to form spheroids having isotropic shapes, or close to them, like human colorectal adenocarcinoma cells (DLD-1). It is more preferable that the third distance is greater than 800 micrometers for cultivation of such cancer cells. On the other hand, it is preferable that the third distance is equal to or smaller than 400 micrometers for cultivation of cancer cells, which are to form spheroids having indefinite shapes, or close to them, like human breast adenocarcinoma cells (SKBR3).

It is preferable that the third distance is equal to or smaller than 400 micrometers for cultivation of normal cells. It is more preferable that the third distance is equal to or smaller than 200 micrometers for cultivation of normal cells.

With regard to the plate, it is preferred that the one first portion and two first portions of the six first portions substantially constitute an equilateral triangle.

With regard to the plate, it is preferred that the two first portions are arranged at positions proximate to each other.

With regard to the plate, it is preferred that the six first portions substantially constitute a regular hexagon.

With regard to the plate, it is preferred that the one first portion and two first portions of the six first portions are substantially included in a regular hexagon.

A plate relating to an aspect of this disclosure includes a member. The member has a first plane and a second plane opposite to the first plane. A plurality of first portions, a second portion, a plurality of third portions, and a fourth portion are formed in the first plane. A first interval between two first portions of the plurality of first portions arranged at positions proximate to each other is different from a second interval between two third positions of the plurality of third positions arranged at positions proximate to each other.

With regard to the plate, it is preferred that each of the two first portions is surrounded by the second portion, and each of the two third portions is surrounded by the fourth portion.

With regard to the plate, it is preferred that at least four first portions of the plurality of first portions are arranged at the first interval from one first portion of the plurality of first portions. At least four third portions of the plurality of third portions are arranged at the second interval from one third portion of the plurality of first portions.

The plate can be used for cultivation of cells to form spheroids. It is preferable that the first interval is equal to or greater than 400 micrometers for cultivation of cancer cells, which are to form spheroids having isotropic shapes, or close to them, like human colorectal adenocarcinoma cells (DLD-1). It is more preferable that the first interval is greater than 800 micrometers for cultivation of such cancer cells. On the other hand, it is preferable that the first interval is equal to or smaller than 400 micrometers for cultivation of cancer cells, which are to form spheroids having indefinite shapes, or close to them, like human breast adenocarcinoma cells (SKBR3).

It is preferable that the first interval is equal to or smaller than 400 micrometers for cultivation of normal cells. It is more preferable that the first interval is equal to or smaller than 200 micrometers for cultivation of normal cells.

With regard to the plate, it is preferred that six first portions of the plurality of first portions are arranged at the first interval from one first portion of the plurality of first portions. At least six third portions of the plurality of third portions are arranged at the second interval from one third portion of the plurality of first portions.

With regard to the plate, it is preferred that the six first portions constitute a regular hexagon.

With regard to the plate, it is preferred that the six third portions constitute a regular hexagon.

With regard to the plate, it is preferred that the six first portions constitute a regular hexagon and the six third portions constitute a regular hexagon.

With regard to the plate, it is preferred that a first affinity of the plurality of first portions to a sample is different from a second affinity of the second portion to the sample. A third affinity of the plurality of third portions to the sample is different from a fourth affinity of the fourth portion to the sample.

Examples of such sample are chemical compound and biological samples such as cell, bacterium, virus and protein.

With regard to the plate, it is preferred that the first affinity is greater than the second affinity, and the third affinity is greater than the fourth affinity.

With regard to the plate, it is preferred that each of the plurality of first portions has a structural object, the shape of which is at least one of a recess and a protrusion.

With regard to the plate, it is preferred that each of the plurality of third portions has a structural object, the shape of which is at least one of a recess and a protrusion.

With regard to the plate, it is preferred that the plurality of first portions and the second portion are formed in a first area, and the plurality of third portions and the fourth portion are formed in a second area.

With regard to the plate, it is preferred that the first area is surrounded by a first fence, and the second area is surrounded by a second fence.

With regard to the plate, it is preferred that the plate further includes a frame and each of the first fence and the second fence is a part of the frame.

With regard to the plate, it is preferred that a plurality of fifth portions and a sixth portion are formed in the first plane. A third interval between two fifth portions of the plurality of fifth portions arranged at positions proximate to each other is different from the first interval and the second interval.

With regard to the plate, it is preferred that the plurality of fifth portions and the sixth portion are surrounded by a third fence.

A method for culturing cells relating to an aspect of this disclosure is carried out using any one of the plates above.

With regard to the method, it is preferred that an interval suitable for effectively forming a spheroid between the first interval and the second interval is examined by the method.

A screening method relating to an aspect of this disclosure is carried out using spheroids formed by a culture of cells by any one of the above methods. The screening method can be applied to examination of pharmacological effects of compounds or drugs.

A method for manufacturing a drug relating to an aspect of this disclosure is carried out based on the results of pharmacological effects examined by the screening method.

### BRIEF DESCRIPTION OF THE DRAWINGS

In the drawings, which illustrate what is currently considered to be the best mode for carrying out the disclosure:

FIG. 1 shows an overview of a plate relating to an aspect of this disclosure and arrangements of affinity regions formed in bottoms of wells of the plate;

FIG. 2 shows arrangements of affinity regions formed in bottoms of wells of a plate relating to an aspect of this disclosure;

FIG. 3 shows a cross-sectional view of a well bottom in which affinity regions are formed;

FIG. 4 shows a cross-sectional view of a well bottom in which recesses are formed;

FIG. 5 shows a cross-sectional view of a well bottom on which protrusions are formed;

FIG. 6 shows spheroids formed by cultivation of DLD-1 using affinity regions, the pitches of which are 400 micrometers, 600 micrometers and 800 micrometers;

FIG. 7 shows spheroids formed by cultivation of SKBR3 using affinity regions, the pitches of which are 400 micrometers, 600 micrometers and 800 micrometers;

FIG. 8 shows time-dependent changes of numbers of cells by cultivation of DLD-1;

FIG. 9 shows time-dependent changes of numbers of cells by cultivation of SKBR3;

FIG. 10 shows outlines of spheroids formed from DLD-1 and SKBR3; and

FIG. 11 shows coefficient of variations (CV) of widths of spheroids formed from DLD-1 and SKBR3.

### DETAILED DESCRIPTION

### Experimental Procedures

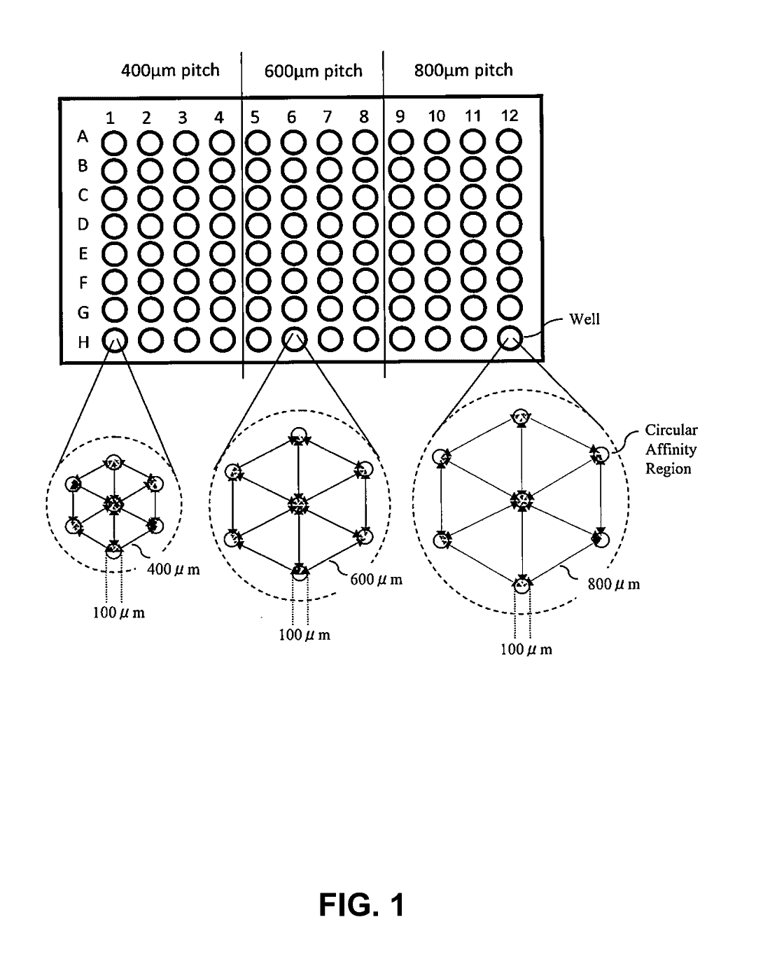

The number of wells can be determined according to the kind of cell or the cultural condition of the cell. An exemplified plate relating to an aspect of this disclosure has 96 wells in total, each of which is surrounded by a black frame as shown in FIG. 1. A portion of the black frame corresponds to an example of the first fence or the second fence explained above. The constituent material of the bottoms of the wells is a resin such as cycloolefin resin, polystyrene, acrylic resin, polyethylene, polypropylene, polycarbonate, polyethylene terephthalate, polyvinyl alcohol and polyvinylidene chloride. Among them, cycloolefin resin is preferable because of its high transparency if the plate is used for imaging. Typically, the thickness of the resin is around 190 micrometers if the constituent material is cycloolefin resin. Plural circular regions, which are 100 micrometers in diameter and have high affinity to biological samples such as cells compared to the periphery of the regions, are formed in the bottom of each of the wells. This enables anchoring and cultivation of biological samples. The density of the regions differs depending on parts of the plate. The circular affinity regions are arranged in three different pitches, i.e., 400-, 600- and 800-micrometer pitches. The numbers of the circular regions per well are 220, 100 and 50 for 400-, 600- and 800-micrometer pitches, respectively.

Three of the circular affinity regions arranged at positions proximate to each other constitute an equilateral triangle. Six of the circular affinity regions arranged at positions proximate to one of the circular affinity regions substantially constitute a regular hexagon.

Alternatively, affinity regions on two straight lines intersecting at an acute angle are arranged at the same distance. Typically, the angle formed by the two straight lines is 60 degrees.

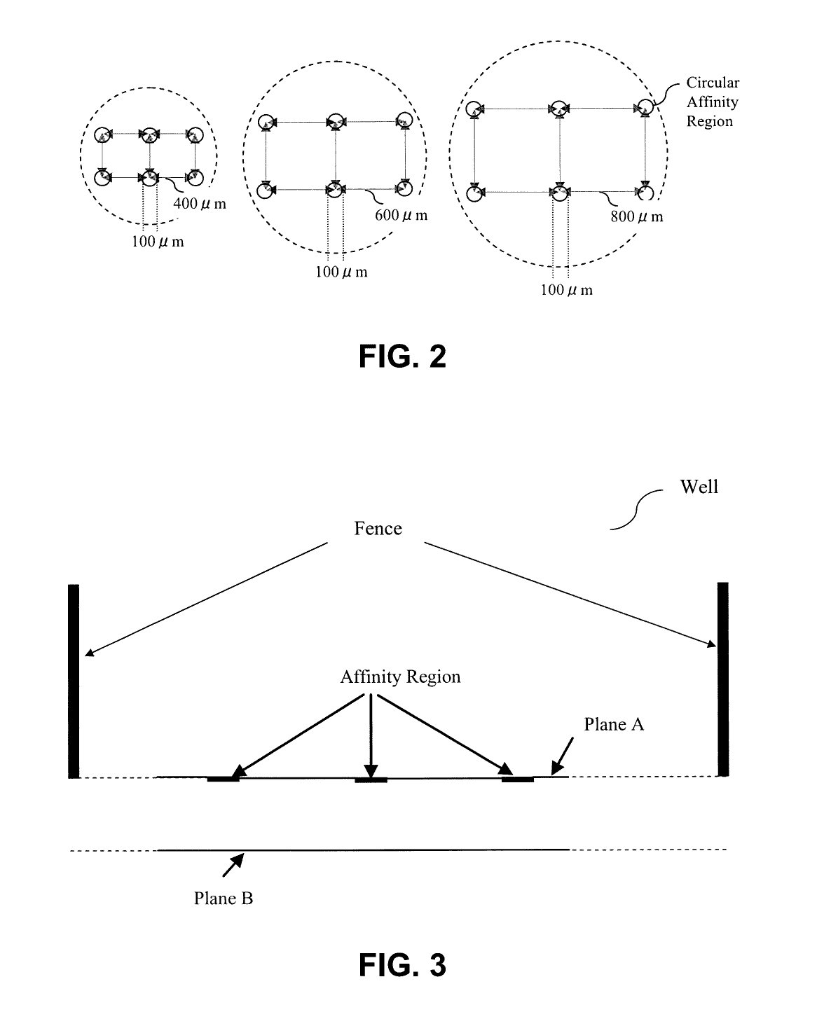

Another plate relating to an aspect of this disclosure has a square arrangement of affinity regions as shown in FIG. 2. Four of the circular affinity regions arranged at positions proximate to each other substantially constitute a square. Alternatively, affinity regions on two straight lines intersecting at a right angle are arranged at the same distance.

FIG. 3 shows a cross-sectional view of the circular affinity regions formed on the well bottom. The affinity regions can be formed by a surface treatment or photolithography technique. The affinity regions are surrounded by a fence contacting plane A. The fence prevents liquid containing a sample dispensed into the well from leaking. The fence may be a portion of the frame. The affinity regions can be formed by formation of hydrophilic portions by using hydrophilic polymer or resin such as polyvinyl alcohol and cellulose.

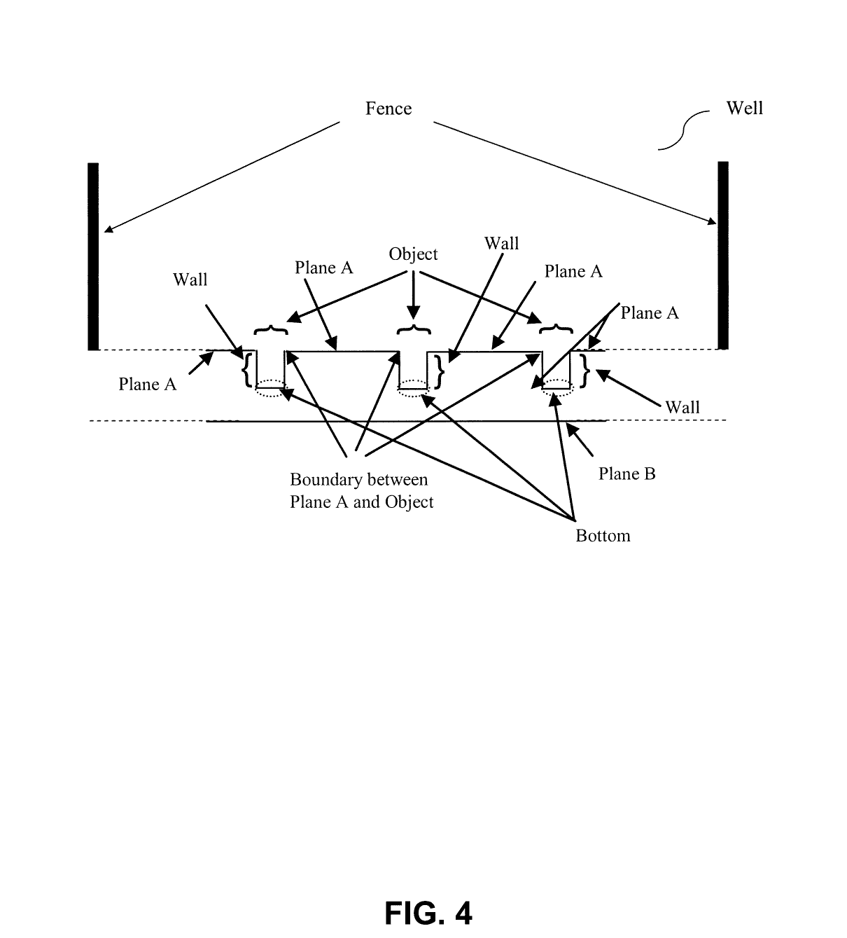

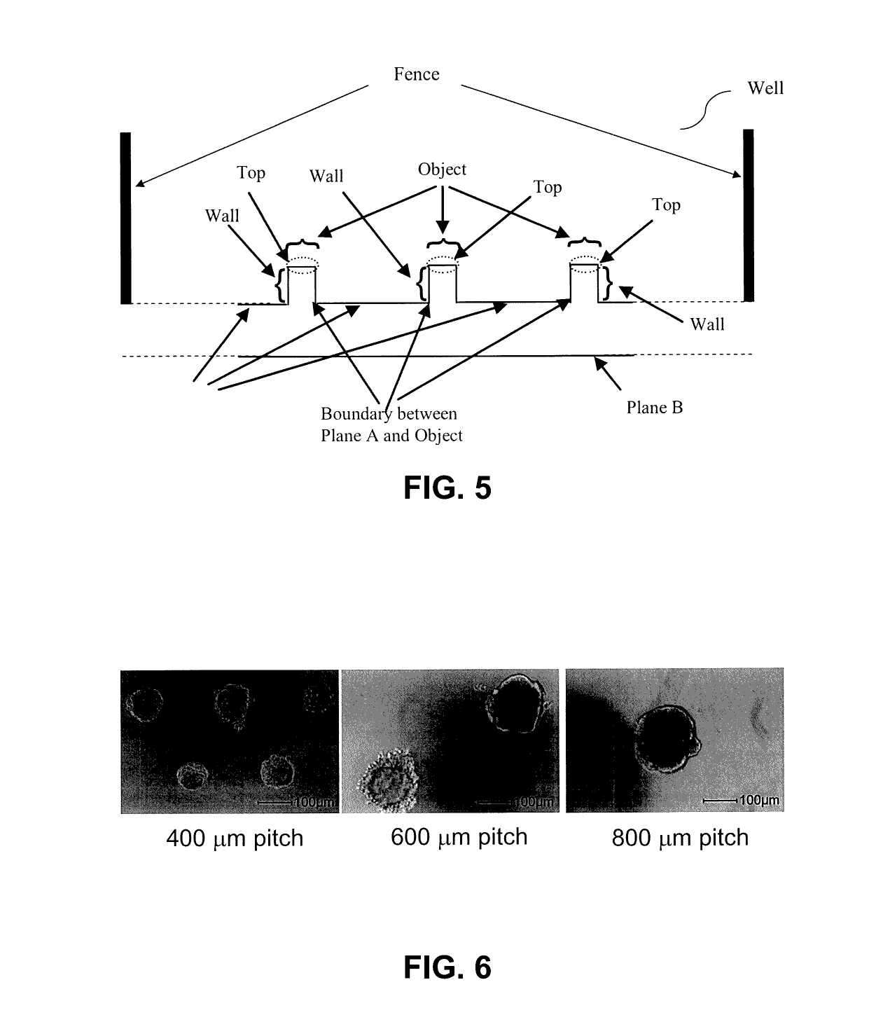

Instead of formation of affinity regions, structural objects such as a recess or protrusion are formed to fix and cultivate biological samples such as cell, bacterium, virus and protein. FIG. 4 shows recesses formed in the bottom of the well explained above while FIG. 5 shows protrusions formed on the bottom of the well.

Each of the recesses is constituted by a bottom and a wall as shown in FIG. 4. The wall extends from a boundary between plane A (the surface of the well bottom) and the recess toward the bottom of the recess. The recesses are surrounded by a fence contacting plane A. The fence prevents liquid containing a sample dispensed into the well from leaking. The fence may be a portion of the frame.

Each of the protrusions is constituted by a top and a wall as shown in FIG. 5. The wall extends from a boundary between plane A and the protrusion toward the top of the protrusion. The protrusions are surrounded by a fence contacting plane A. The fence prevents liquid containing a sample dispensed into the well from leaking. The fence may be a portion of the frame.

The surface conditions of at least one of the wall and the bottom or the top can be adjusted by a surface treatment or selection of material used for the wall and the bottom or the top.

Cultivations of human colorectal adenocarcinoma cells DLD-1 and human breast adenocarcinoma cells SKBR3 were carried out using the plate as shown in FIG. 1.

Each of two suspensions containing human colorectal adenocarcinoma cells DLD-1 and human breast adenocarcinoma cells SKBR3, respectively, was dispensed into wells of the plate in which the circular affinity regions are arranged in the three different pitches, i.e., 400-, 600- and 800-micrometer pitches.

Typical numbers of cells to be seeded for one well are 2,800-5,600, 1,200-2,400 and 650-1,300 for 400-, 600- and 800-micrometer pitches, respectively. On this occasion, the numbers of cells seeded in each of the wells were 2,800, 1,200 and 650 for 400-, 600- and 800-micrometer pitches, respectively. The seeded cells were incubated in the presence of 5% humidified CO₂ at 37 degrees Celsius. The incubation medium is replaced with fresh medium every 1 to 3 days.

Spheroids formed from DLD-1 on the well bottoms having affinity regions with 400-, 600- and 800-micrometer pitches after 14 days of culture are shown in FIG. 6. Apparent formations of spheroids were observed for all of 400-, 600- and 800-micrometer pitches. This indicates that culture of biological samples on a surface with affinity regions constituting regular hexagons or equilateral triangles is effective for formation of spheroids.

The spheroid formed on the well bottom having affinity regions of an 800-micrometer pitch was the largest in size among the spheroids formed on the well bottoms having affinity regions with the three pitches.

FIG. 8 shows time-dependent changes of numbers of cells by cultivation of DLD-1 on the well bottoms having affinity regions with the three pitches. The changes of numbers of cells were observed by detecting changes of amounts of luminescence emitted from the spheroids. Remarkable growth of spheroids was observed for the 800-micrometer pitch, even after 10 days of culture.

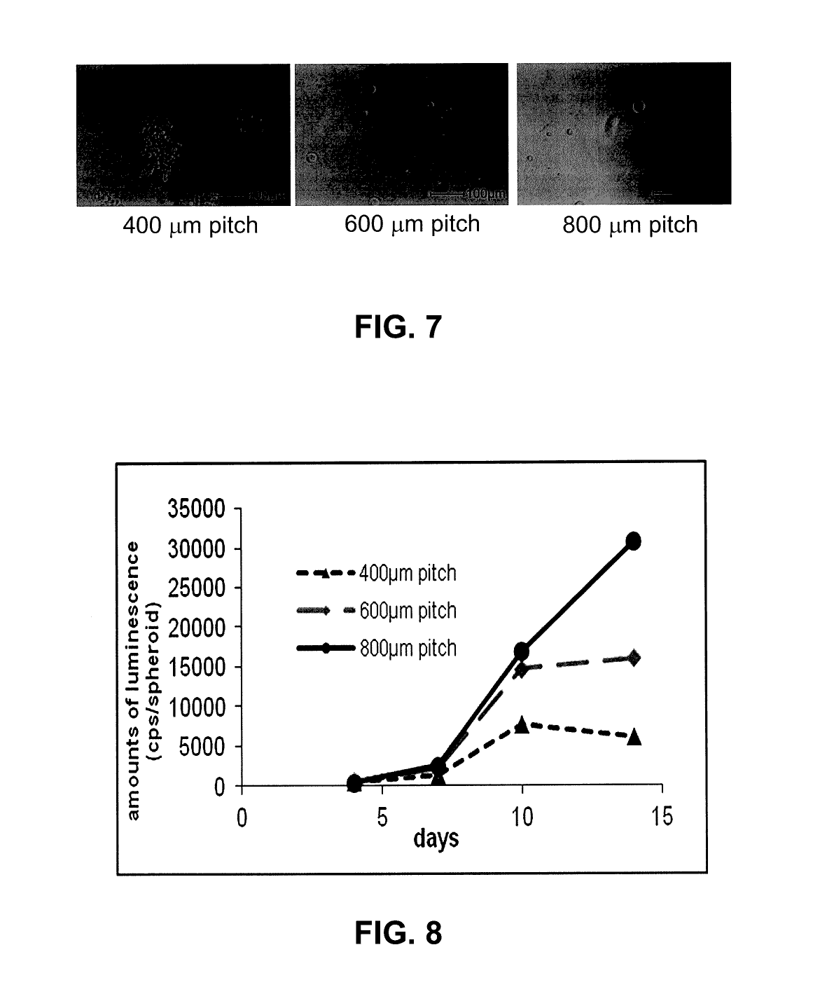

Spheroids formed from SKBR3 on the well bottoms having affinity regions with 400-, 600- and 800-micrometer pitches after 14 days of culture are shown in FIG. 7. Apparent formation of spheroids was observed for the 400-micrometer pitch, while apparent formations of spheroids were not observed for the 600- and 800-micrometer pitches under this condition.

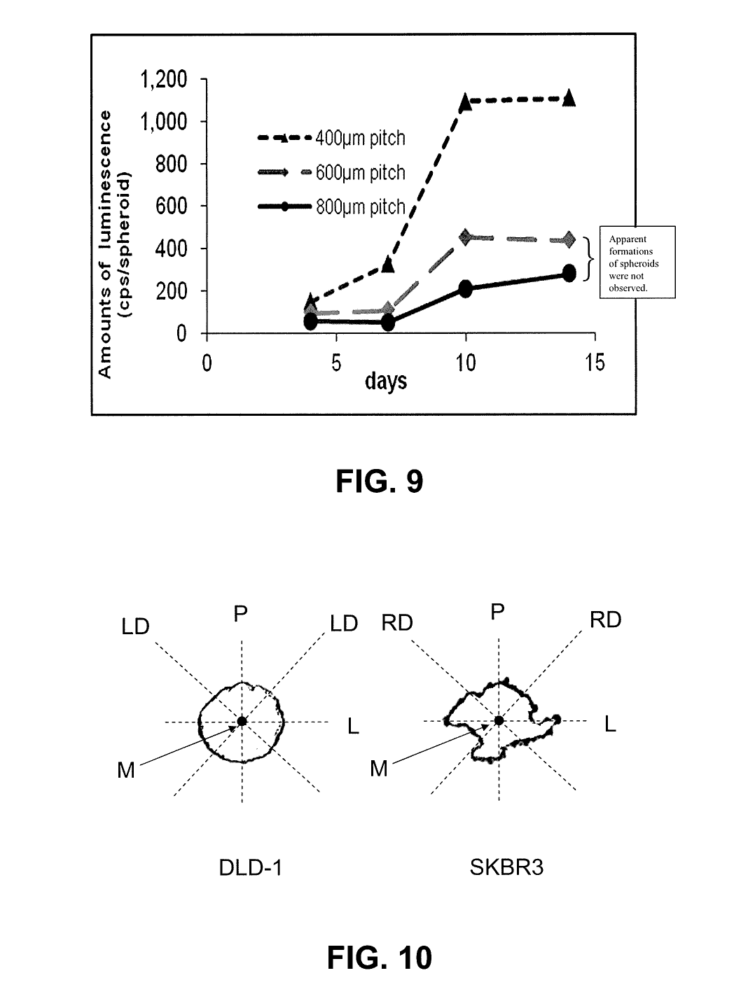

FIG. 9 shows time-dependent changes of numbers of cells by cultivation of SKBR3 on the well bottoms having affinity regions with the three pitches. The changes of numbers of cells were observed by detecting changes of amounts of luminescence emitted from the spheroids. The growth of spheroids on the well bottom having affinity regions of the 400-micrometer pitch stagnated after 10 days of culture.

FIG. 10 shows outlines of spheroids formed from DLD-1 and SKBR3 and axes of length measurements of spheroids. The spheroid formed from DLD-1 is subglobular, while the spheroid formed from SKBR3 has an irregular shape as seen in FIG. 10. Axis L indicates a line including the maximum length of the spheroid. Axis L indicates a line that passes through the midpoint (M) of the maximum length and is perpendicular to the Axis L. Axes RD and LD are lines that pass through midpoint M and intersect with Axis L at 45 and 135 degrees, respectively. The distance between points at the intersections of each of the four axes with the outlines is gauged.

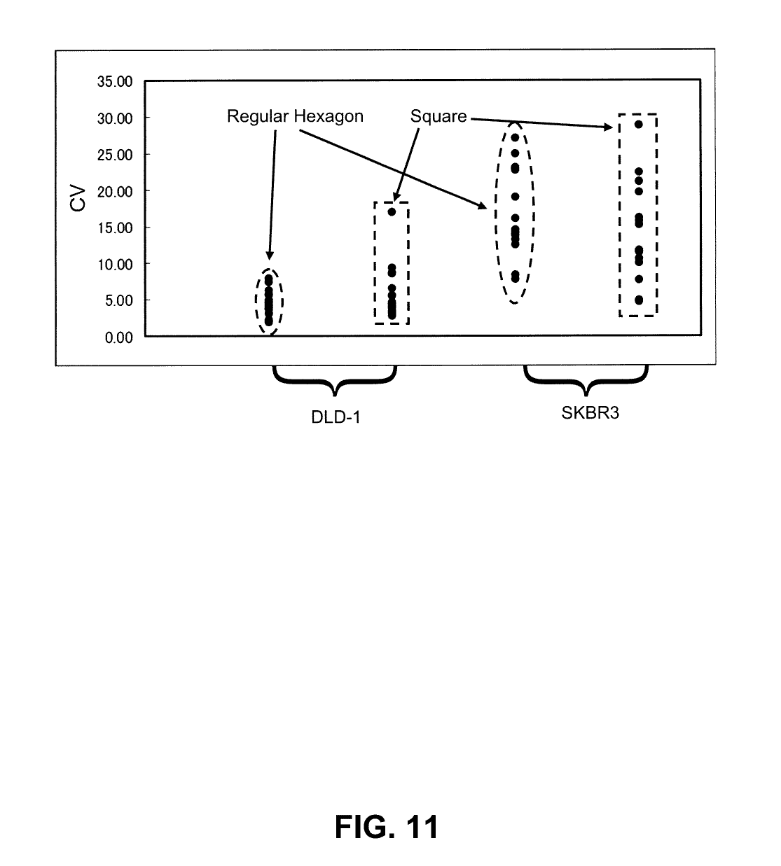

FIG. 11 shows coefficients of variations (CV) of widths of spheroids formed from DLD-1 and SKBR3 measured by the foregoing method. All of the spheroids were formed on the well bottom having affinity regions, the pitch of which is 400 micrometers.

Coefficients of variation of the distances gauged using the four axes is estimated for each of 15 spheroids formed from DLD-1 on the well bottom having affinity regions constituting regular hexagons.

Coefficients of variation of the distances gauged using the four axes is estimated for each of 15 spheroids formed from DLD-1 on the well bottom having affinity regions constituting squares.

Coefficients of variation of the distances gauged using the four axes is estimated for each of 15 spheroids formed from SKBR3 on the well bottom having affinity regions constituting regular hexagons.

Coefficients of variation of the distances gauged using the four axes is estimated for each of 15 spheroids formed from SKBR on the well bottom having affinity regions constituting squares.

The coefficients of variation regarding the spheroids formed from DLD-1 on the well bottom with regular hexagonal pitch range from 1.86 to 7.89, while the coefficients of variation regarding the spheroids formed from DLD-1 on the well bottom with square pitch range from 2.71 to 17.05.

Cells that form spheroids, the sizes of which show a CV of 1.86-17.05, can be categorized in cells having an isotropic shape explained above. More specifically, cells that form spheroids, the sizes of which show a CV of 1.86-7.89, can be categorized in cells having an isotropic shape.

In other words, the spheroids formed for DLD-1 on the well bottom with a regular hexagonal pitch have a tendency to be more subglobular compared to those formed from DLD-1 on the well bottom with a square pitch.

The coefficients of variation regarding the spheroids formed from SKBR3 on the well bottom with a regular hexagonal pitch range from 7.80 to 24.95, while the coefficients of variation regarding the spheroids formed from SKBR3 on the well bottom with a square pitch range from 4.87 to 28.81.

Cells that form spheroids, the sizes of which show a CV of 4.87-28.81, can be categorized in cells having an indefinite shape explained above. More specifically, cells that form spheroids, the sizes of which show a CV of 7.80-24.95, can be categorized in cells having an indefinite shape.

The spheroids formed from SKBR3 essentially have a tendency to have irregular shapes. In other words, the plates relating to an aspect of this disclosure enable forming spheroids from cells having irregular shapes such as SKBR3.

A plate having affinity regions or structural objects of differing pitches from each other as shown in FIG. 1 or FIG. 2 is especially of great utility, even for cells having irregular shapes, because an optimum pitch of the affinity regions or structural objects for can be determined by minimum trials.

In response to cell type, an optimum plate or pitch of affinity regions or structural objects is determined. Cell type can be determined by parameters relating to size or shape as explained above.

In one instance, a cell with a coefficient of variation of distances obtained based on the four axes explained above is equal to or greater than 10 can be judged to have an irregular shape.

1. A plate, comprising a member, wherein:
the member has a first plane, a second plane and a plurality of structural objects, each of which has at least one of a bottom in a recess and a top in a protrusion;
the plurality of structural objects are formed in the first plane;
a first distance from the second plane to a boundary between each of the plurality of structural objects and the first plane is different from a second distance between the second plane and the at least one of the bottom and the top;
at least five structural objects of the plurality of structural objects are arranged at a first interval from one structural object of the plurality of structural objects;
an affinity of a first portion being the at least one of a bottom in a recess and a top in a protrusion to a sample is different from an affinity of a second portion being a part of an area other than the first portion to the sample; and
wherein the one structural object of the plurality of structural objects and the at least five structural objects of the plurality of structural objects are surrounded by a fence.

2. The plate of claim 1, wherein six structural objects of the plurality of structural objects including the at least five structural objects are arranged at the first interval from the one structural object.

3. The plate of claim 2, wherein the one structural object and two structural objects of the six structural objects substantially constitute an equilateral triangle.

4. The plate of claim 3, wherein the two structural objects are arranged at positions proximate to each other.

5. The plate of claim 2, wherein the six structural objects of the plurality of structural objects substantially constitute a regular hexagon.

6. The plate of claim 5, wherein the one structural object and the two structural objects of the six structural objects of the plurality of structural objects are substantially included in the regular hexagon.

7-12. (canceled)

13. -A- The plate of claim 1, wherein:
the member has a plurality of first portions, a second portion, a plurality of third portions and a fourth portion in the first plane;
a second interval between two third positions of the plurality of third positions arranged at positions proximate to each other is different from the first interval; and
an affinity of the third portion to the sample is different from an affinity of the fourth portion to the sample.

14. The plate of claim 13, wherein:
each of the two first portions is surrounded by the second portion; and
each of the two third portions is surrounded by the fourth portion.

15. The plate of claim 13, wherein:
at least four third portions of the plurality of third portions are arranged at the second interval from one third portion of the plurality of first portions.

16. The plate of claim 13, wherein:
at least six third portions of the plurality of third portions are arranged at the second interval from one third portion of the plurality of first portions.

17-21. (canceled)

22. The plate of claim 13, wherein:
an affinity of the first portion to the sample is greater than an affinity of the second portion to the sample;
an affinity of the third portion to the sample is greater than an affinity of the fourth portion to the sample;
the plurality of first portions and the second portion are formed in a first area; and
the plurality of third portions and the fourth portion are formed in a second area.

23. The plate of claim 22, wherein:
the first area is surrounded by a first fence; and
the second area is surrounded by a second fence.

24. The plate of claim 22, further comprising:
a frame,
wherein each of the first fence and the second fence is a part of the frame.

25-26. (canceled)

27. A method for culturing cells, wherein the method is carried out utilizing the plate of claim 1.

28. The method of claim 27, wherein an interval suitable for effectively forming a spheroid between the first interval and the second interval is examined by the method.

29. The method according to claim 27, further comprising:
forming spheroids with the culture and
screening utilizing said spheroids.

30. The plate claim 1, wherein the sample is any one of a chemical compound and a biological sample.

31. The plate of claim 30, wherein the sample comprises a cell, a bacterium, a virus or a protein.

32. The plate of claim 1, wherein the first portion has a circular region of about 100 micrometers in diameter.

33. The plate of claim 1, wherein the first portion is formed as a hydrophilic portion utilizing hydrophilic polymer.
